(19) United States
(12) Patent Application Publication
(10) Pub. No.: US 2004/0229255 A1
(43) Pub. Date: Nov. 18, 2004

(54) Nucleotide sequence coding for the OtsA protein

(75) Inventors: Thomas Hermann, Bielefeld (DE); Andreas Wolf, Koeln (DE); Susanne Morbach, Juelich (DE); Reinhard Kraemer, Juelich (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/801,847

(22) Filed: Mar. 17, 2004

Related U.S. Application Data
(62) Division of application No. 10/058,945, filed on Jan. 30, 2002.

(30) Foreign Application Priority Data
Jan. 30, 2001 (DE) 101 03 873.9
Mar. 7, 2001 (DE) 101 10 760.9

(51) Int. Cl. C12Q 1/68; C07H 21/04; C12P 13/00; C12N 9/12; C12N 1/21; C12N 15/74

(52) U.S. Cl. 435/6; 435/69.1; 435/252.3; 435/320.1; 536/23.2; 435/194

(57) ABSTRACT

The present invention provides nucleotide sequences from Coryneform bacteria which code for the OtsA protein and processes for the fermentative preparation of amino acids using bacteria in which the otrA gene is attenuated.

### BACKGROUND OF THE INVENTION

1. Field of the Invention

The present invention provides nucleotide sequences from Coryneform bacteria which code for the OtsA protein and a process for the fermentative preparation of amino acids using bacteria in which the otsA gene is attenuated.

2. Discussion of the Background

L-Amino acids, in particular L-lysine, are used in human medicine and in the pharmaceuticals industry, in the foodstuffs industry and very particularly in animal nutrition.

It is known that amino acids are prepared by fermentation from strains of coryneform bacteria, in particular Corynebacterium glutamicum. Because of their great importance, work is constantly being undertaken to improve the preparation processes. Improvements to the process can relate to fermentation measures, such as, for example, stirring and supply of oxygen, or the composition of the nutrient media, such as, for example, the sugar concentration during the fermentation, or the working up to the product form by, for example, ion exchange chromatography, or the intrinsic output properties of the microorganism itself.

Methods of mutagenesis, selection and mutant selection are used to improve the output properties of these microorganisms. Strains which are resistant to antimetabolites or are auxotrophic for metabolites of regulatory importance and which produce amino acids are obtained in this manner.

Methods of the recombinant DNA technique have also been employed for some years for improving the strain of Corynebacterium strains which produce L-amino acid, by amplifying individual amino acid biosynthesis genes and investigating the effect on the amino acid production.

However, there remains a critical need for improved methods of producing L-amino acids and thus for the provision of strains of bacteria producing higher amounts of L-amino acids. On a commercial or industrial scale even small improvements in the yield of L-amino acids, or the efficiency of their production, are economically significant. Prior to the present invention, it was not recognized that enhancing the otsA gene encoding the OtsA trehalose 6-phosphate synthase would improve L-amino acid yields.

### SUMMARY OF THE INVENTION

One object of the present invention is providing a new process adjuvant for improving the fermentative production of L-amino acids, particularly L-lysine. Such process adjuvants include enhanced bacteria, preferably enhanced Coryneform bacteria which express attenuated levels of trehalose 6-phosphate synthase, which is encoded by the otsA gene.

Thus, another object of the present invention is providing such a bacterium, which expresses attenuated amounts of trehalose 6-phosphate synthase or gene products of the otsA gene.

Another object of the present invention is providing a bacterium, preferably a Coryneform bacterium, which expresses a polypeptide that has attenuated trehalose 6-phosphate activity.

Another object of the invention is to provide a nucleotide sequence encoding a polypeptide which has a OtsA trehalose 6-phosphate synthase sequence. One embodiment of such a sequence is the nucleotide sequence of SEQ ID NO: 1.

A further object of the invention is a method of making a trehalose 6-phosphate synthase or an isolated polypeptide having trehalose 6-phosphate synthase activity, as well as use of such isolated polypeptides in the production of amino acids. One embodiment of such a polypeptide is the polypeptide having the amino acid sequence of SEQ ID NO: 2.

In one embodiment the invention provides isolated polypeptides comprising the amino acid sequence in SEQ ID NOS:2.

Other objects of the invention include methods of detecting nucleic acid sequences homologous to SEQ ID NO: 1, particularly nucleic acid sequences encoding polypeptides that have the trehalose 6-phosphate synthase activity, and methods of making nucleic acids encoding such polypeptides.

The above objects highlight certain aspects of the invention. Additional objects, aspects and embodiments of the invention are found in the following detailed description of the invention.

### BRIEF DESCRIPTION OF THE DRAWINGS

FIG. 1: Plasmid pUC18otsA.

FIG. 2: Plasmid pK19mobsacBΔotsA.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Cold Spring Harbor Laboratory, New York (1989), Current Protocols in Molecular Biology, Ausebel et al (eds), John Wiley and Sons, Inc. New York (2000)and the various references cited therein.

“L-amino acids” or “amino acids” as used herein mean one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine is particularly preferred.

When L-lysine or lysine are mentioned in the following, not only the bases but also the salts, such as e.g. lysine monohydrochloride or lysine sulfate, are meant by this.

The invention provides an isolated polynucleotide from coryneform bacteria, comprising a polynucleotide sequence which codes for the otsA gene, chosen from the group consisting of

a) polynucleotide which is identical to the extent of at least 70% to a polynucleotide which codes for a polypeptide which comprises the amino acid sequence of SEQ ID No. 2,

b) polynucleotide which codes for a polypeptide which comprises an amino acid sequence which is identical to the extent of at least 70% to the amino acid sequence of SEQ ID No. 2,

c) which is complementary to the polynucleotides of a) or b),

d) polynucleotide comprising at least 15 successive nucleotides of the polynucleotide sequence of a), b) or c),

the polypeptide preferably having the activity of trehalose 6-phosphate synthase.

The invention also provides the above-mentioned polynucleotide, this preferably being a DNA which is capable of replication, comprising:

(i) the nucleotide sequence, shown in SEQ ID No.1, or

(ii) at least one sequence which corresponds to sequence (i) within the degeneracy of the genetic code, or

(iii) at least one sequence which hybridizes with the sequences complementary to sequences (i) or (ii), and optionally

(iv) sense mutations of neutral function in (i) which do not modify the activity of the protein/polypeptide.

Finally, the invention also provides polynucleotides chosen from the group consisting of

a) comprising at least 15 successive nucleotides chosen from the nucleotide sequence of SEQ ID No. 1 between positions 1 and 883,

b) polynucleotides comprising at least 15 successive nucleotides chosen from the nucleotide sequence of SEQ ID No. 1 between positions 884 and 2338,

c) polynucleotides comprising at least 15 successive nucleotides chosen from the nucleotide sequence of SEQ ID No. 1 between positions 2339 and 3010.

The invention also provides:

a polynucleotide, in particular DNA, which is capable of replication and comprises the nucleotide sequence as shown in SEQ ID No.1;

a polynucleotide which codes for a polypeptide which comprises the amino acid sequence as shown in SEQ ID No. 2;

a vector containing parts of the polynucleotide according to the invention, but at least 15 successive nucleotides of the sequence claimed,

and coryneform bacteria in which the otsA gene is attenuated, in particular by an insertion or deletion.

The invention also provides polynucleotides, which substantially comprise a polynucleotide sequence, which are obtainable by screening by means of hybridization of a corresponding gene library of a coryneform bacterium, which comprises the complete gene or parts thereof, with a probe which comprises the sequence of the polynucleotide according to the invention according to SEQ ID No.1 or a fragment thereof, and isolation of the polynucleotide sequence mentioned.

Polynucleotides which comprise the sequences according to the invention are suitable as hybridization probes for RNA, cDNA and DNA, in order to isolate, in the full length, nucleic acids or polynucleotides or genes which code for trehalose 6-phosphate synthase or to isolate those nucleic acids or polynucleotides or genes which have a high similarity with the sequence of the otsA gene. They are also suitable for incorporation into so-called “arrays”, “micro arrays” or “DNA chips” in order to detect and determine the corresponding polynucleotides.

Polynucleotides which comprise the sequences according to the invention are furthermore suitable as primers with the aid of which DNA of genes which code for trehalose 6-phosphate synthase can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides with a length of at least 31, 32, 33, 34, 35, 36, 37, 38, 39 or 40 or at least 41, 42, 43, 44, 45, 46, 47, 48, 49 or 50 nucleotides are also suitable. oligonucleotides with a length of at least 100, 150, 200, 250 or 300 nucleotides are optionally also suitable.

“Isolated” means separated out of its natural environment.

“Polynucleotide” in general relates to polyribonucleotides and polydeoxyribonucleotides, it being possible for these to be non-modified RNA or DNA or modified RNA or DNA.

The polynucleotides according to the invention include a polynucleotide according to SEQ ID No. 1 or a fragment prepared therefrom and also those which are at least 70% to 80%, preferably at least 81% to 85%, particularly preferably at least 86% to 90% and very particularly referably at least 91%, 93%, 95%, 97% or 99% identical to he polynucleotide according to SEQ ID No. 1 or a fragment repared therefrom.

“Polypeptides” are understood as meaning peptides or proteins which comprise two or more amino acids bonded via peptide bonds.

The polypeptides according to the invention include a polypeptide according to SEQ ID No. 2, in particular those with the biological activity of trehalose 6-phosphate synthase, and also those which are at least 70% to 80%, preferably at least 81% to 85%, particularly preferably at least 86% to 90% and very particularly preferably at least 91%, 93%, 95%, 97% or 99% identical to the polypeptide according to SEQ ID No. 2 and have the activity mentioned.

The invention furthermore relates to a process for the fermentative preparation of amino acids chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using coryneform bacteria which in particular already produce amino acids and in which the nucleotide sequences which code for the otsA gene are attenuated, in particular eliminated or expressed at a low level.

The term “attenuation” in this connection describes the reduction or elimination of the intracellular activity of one or more enzymes or proteins in a microorganism which are coded by the corresponding DNA, for example by using a weak promoter or using a gene or allele which codes for a corresponding enzyme or protein with a low activity or inactivates the corresponding gene or enzyme (protein) and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

Preferably, a bacterial strain with attenuated expression of the otsA gene encoding a trehalose 6-phosphate synthase will improve amino acid yield at least 1%.

The microorganisms provided by the present invention can prepare amino acids from glucose, sucrose, lactose, fructose, maltose, molasses, starch, cellulose or from glycerol and ethanol. They can be representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum (C. glutamicum), are in particular the known wild-type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and L-amino acid-producing mutants or strains prepared therefrom, such as, for example, the L-lysine-producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464

Corynebacterium glutamicum DM58-1

Corynebacterium glutamicum DG52-5

Corynebacterium glutamicum DSM5715 and

Corynebacterium glutamicum DSM12866.

The new otsA gene from C. glutamicum which codes for the enzyme trehalose 6-phosphate synthase (EC 2.4.1.15) has been isolated.

To isolate the otsA gene or also other genes of C. glutamicum, a gene library of this microorganism is first set up in Escherichia coli (E. coli). The setting up of gene libraries is described in generally known textbooks and handbooks. The textbook by Winnacker: Gene und Klone, Eine Einführung in die Gentechnologie (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in λ vectors by Kohara et al. (Cell 50, 495 -508 (1987)). Bathe et al. (Molecular and General Genetics, 252:255-265, 1996) describe a gene library of C. glutamicum ATCC13032, which was set up with the aid of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:1563-1575).

Börmann et al. (Molecular Microbiology 6(3), 317-326)) (1992)) in turn describe a gene library of C. glutamicum ATCC13032 using the cosmid pHC79 (Hohn and Collins, 1980, Gene 11, 291-298).

To prepare a gene library of C. glutamicum in E. coli it is also possible to use plasmids such as pBR322 (Bolivar, 1979, Life Sciences, 25, 807-818) or pUC9 (Vieira et al., 1982, Gene, 19:259-268). Suitable hosts are, in particular, those E. coli strains which are restriction- and recombination-defective, such as, for example, the strain DH5αmcr, which has been described by Grant et al. (Proceedings of the National Academy of Sciences USA, 87 (1990) 4645-4649). The long DNA fragments cloned with the aid of cosmids or other λ vectors can then in turn be subcloned and subsequently sequenced in the usual vectors which are suitable for DNA sequencing, such as is described e. g. by Sanger et al. (Proceedings of the National Academy of Sciences of the United States of America, 74:5463-5467, 1977).

The resulting DNA sequences can then be investigated with known algorithms or sequence analysis programs, such as e.g. that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum which codes for the otsA gene and which, as SEQ ID No. 1, is a constituent of the present invention has been found. The amino acid sequence of the corresponding protein has furthermore been derived from the present DNA sequence by the methods described above. The resulting amino acid sequence of the otsA gene product is shown in SEQ ID No. 2. It is known that enzymes endogenous in the host can split off the N-terminal amino acid methionine or formylmethionine of the protein formed.

Coding DNA sequences which result from SEQ ID No. 1 by the degeneracy of the genetic code are also a constituent of the invention. In the same way, DNA sequences which hybridize with SEQ ID No. 1 or parts of SEQ ID No. 1 are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. Such mutations are also called, inter alia, neutral substitutions. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences which result in a corresponding manner from SEQ ID No. 2 are also a constituent of the invention.

In the same way, DNA sequences which hybridize with SEQ ID No. 1 or parts of SEQ ID No. 1 are a constituent of the invention. Finally, DNA sequences which are prepared by the polymerase chain reaction (PCR) using primers which result from SEQ ID No. 1 are a constituent of the invention. Such oligonucleotides typically have a length of at least 15 nucleotides.

Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, that is to say only hybrids in which the probe and target sequence, i. e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50° C.-68° C., for example, can be employed for the hybridization reaction. Probes can also hybridize here with polynucleotides which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50° C.-68° C. being established. It is optionally possible to lower the salt concentration to 0.1×SSC. Polynucleotide fragments which are, for example, at least 70% or at least 80% or at least 90% to 95% or at least 96% to 99% identical to the sequence of the probe employed can be isolated by increasing the hybridization temperature stepwise from 50° C. to 68° C. in steps of approx. 1-2° C. It is also possible to isolate polynucleotide fragments which are completely identical to the sequence of the probe employed. Further instructions on hybridization are obtainable on the market in the form of so-called kits (e.g. DIG Easy Hyb from Roche Diagnostics GmbH, Mannheim, Germany, Catalogue No. 1603558).

Instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

It has been found that coryneform bacteria produce amino acids in an improved manner after attenuation of the otsA gene.

To achieve an attenuation, either the expression of the otsA gene or the catalytic/regulatory properties of the enzyme protein can be reduced or eliminated. The two measures can optionally be combined.

The reduction in gene expression can take place by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in the patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1996)), Vasicova et al. (Journal of Bacteriology 181: 6188 (1999)) and in known textbooks of genetics and molecular biology, such as e.g. the textbook by Knippers (“Molekulare Genetik”, 6th edition, Georg Thieme Verlag, Stuttgart, Germany, 1995) or that by Winnacker (“Gene und Klone”, VCH Verlagsgesellschaft, Weinheim, Germany, 1990).

Mutations which lead to a change or reduction in the catalytic properties of enzyme proteins are known from the prior art; examples which may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms”, Reports from the Jülich Research Center, Jül -2906, ISSN09442952, Jülich, Germany, 1994). Summarizing descriptions can be found in known textbooks of genetics and molecular biology, such as e.g. that by Hagemann (“Allgemeine Genetik”, Gustav Fischer Verlag, Stuttgart, 1986).

Possible mutations are transitions, transversions, insertions and deletions. Depending on the effect of the amino acid exchange on the enzyme activity, “missense mutations” or “nonsense mutations” are referred to. Insertions or deletions of at least one base pair (bp) in a gene lead to frame shift mutations, as a consequence of which incorrect amino acids are incorporated or translation is interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik”, 6th edition, Georg Thieme Verlag, Stuttgart, Germany, 1995), that by Winnacker (“Gene und Klone”, VCH Verlagsgesellschaft, Weinheim, Germany, 1990) or that by Hagemann (“Allgemeine Genetik”, Gustav Fischer Verlag, Stuttgart, 1986).

A common method of mutating genes of C. glutamicum is the method of “gene disruption” and “gene replacement” described by Schwarzer and Pühler (Bio/Technology 9, 84-87 (1991)).

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega Corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the central part of the coding region of the gene is then transferred into the desired strain of C. glutamicum by conjugation or transformation. The method of conjugation is described, for example, by Schäfer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) to eliminate the recA gene of C. glutamicum.

In the method of “gene replacement”, a mutation, such as e.g. a deletion, insertion or base exchange, is established in vitro in the gene of interest. The allele prepared is in turn cloned in a vector which is not replicative for C. glutamicum and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch et al. (Microbiology 144, 915-927 (1998)) to eliminate the pyc gene of C. glutamicum by a deletion.

A deletion, insertion or a base exchange can be incorporated into the otsA gene in this manner.

In addition, it may be advantageous for the production of L-amino acids to enhance, in particular over-express, one or more enzymes of the particular biosynthesis pathway, of glycolysis, of anaplerosis, of the citric acid cycle, of the pentose phosphate cycle, of amino acid export and optionally regulatory proteins, in addition to the attenuation of the otsA gene.

The term “enhancement” in this connection describes the increase in the intracellular activity of one or more enzymes (proteins) in a microorganism which are coded by the corresponding DNA, for example by increasing the number of copies of the gene or of the genes or alleles, using a potent promoter or using a gene or allele which codes for a corresponding enzyme having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Thus, for the preparation of L-lysine, in addition to the attenuation of the otsA gene at the same time one or more of the genes chosen from the group consisting of

the dapA gene which codes for dihydrodipicolinate synthase (EP-B 0 197 335),

the gap gene which codes for glyceraldehyde 3-phosphate dehydrogenase (Eikmanns (1992), Journal of Bacteriology 174:6076-6086),

the eno gene which codes for enolase (DE: 19947791.4),

the tpi gene which codes for triose phosphate isomerase (Eikmanns (1992), Journal of Bacteriology 174:6076-6086),

the pgk gene which codes for 3-phosphoglycerate kinase (Eikmanns (1992), Journal of Bacteriology 174:6076-6086),

the zwf gene which codes for glucose 6-phosphate dehydrogenase (JP-A-09224661),

the pyc gene which codes for pyruvate carboxylase (DE-A-198 31 609),

the mqo gene which codes for malate-quinone oxidoreductase (Molenaar et al., European Journal of Biochemistry 254, 395-403 (1998)),

the lysC gene which codes for a feed-back resistant aspartate kinase (Accession No.P26512; EP-B-0387527; EP-A-0699759; WO 00/63388),

the lysE gene which codes for lysine export (DE-A-195 48 222),

the zwa1 gene which codes for the Zwa1 protein (DE: 19959328.0, DSM 13115)

can be enhanced, in particular over-expressed.

It may be furthermore advantageous for the production of L-lysine, in addition to the attenuation of the otsA gene, at the same time for one or more of the genes chosen from the group consisting of

the pck gene which codes for phosphoenol pyruvate carboxykinase (DE 199 50 409.1, DSM 13047),

the pgi gene which codes for glucose 6-phosphate isomerase (U.S. Ser. No. 09/396,478, DSM 12969),

the poxB gene which codes for pyruvate oxidase (DE:1995 1975.7, DSM 13114),

the zwa2 gene which codes for the Zwa2 protein (DE: 19959327.2, DSM 13113),

the fda gene which codes for fructose 1,6-bisphosphate aldolase (Accession No. X17313; von der Osten et al., Molecular Microbiology 3 (11), 1625-1637 (1989)),

the hom gene which codes for homoserine dehydrogenase (EP-A -0131171),

the thrB gene which codes for homoserine kinase (Peoples, O. W., et al., Molecular Microbiology 2 (1988): 63-72) and

the panD gene which codes for aspartate decarboxylase (EP-A-1006192),

to be attenuated, in particular for the expression thereof to be reduced.

The attenuation of homoserine dehydrogenase can also be achieved, inter alia, by amino acid exchanges, such as, for example, by exchange of L-valine for L-alanine, L-glycine or L-leucine in position 59 of the enzyme protein, by exchange of L-valine by L-isoleucine, L-valine or L-leucine in position 104 of the enzyme protein and/or by exchange of L-asparagine by L-threonine or L-serine in position 118 of the enzyme protein.

The attenuation of homoserine kinase can also be achieved, inter alia, by amino acid exchanges, such as, for example, by exchange of L-alanine for L-valine, L-glycine or L-leucine in position 133 of the enzyme protein and/or by exchange of L-proline by L-threonine, L-isoleucine or L-serine in position 138 of the enzyme protein.

The attenuation of aspartate decarboxylase can also be achieved, inter alia, by amino acid exchanges, such as, for example, by exchanges of L-alanine for L-glycine, L-valine or L-isoleucine in position 36 of the enzyme protein.

In addition to the attenuation of the otsA gene it may furthermore be advantageous for the production of amino acids to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of L-amino acids. A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as, for example, soya oil, sunflower oil, groundnut oil and coconut fat, fatty acids, such as, for example, palmitic acid, stearic acid and linoleic acid, alcohols, such as, for example, glycerol and ethanol, and organic acids, such as, for example, acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as, for example, magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as, for example, fatty acid polyglycol esters, can be employed to control the development of foam. Suitable substances having a selective action, such as, for example, antibiotics, can be added to the medium to maintain the stability of plasmids. To maintain aerobic conditions, oxygen or oxygen-containing gas mixtures, such as, for example, air, are introduced into the culture. The temperature of the culture is usually 20° C. to 45° C., and preferably 25° C to 40° C. Culturing is continued until a maximum of the desired product has formed. This target is usually reached within 10 hours to 160 hours.

Methods for the determination of L-amino acids are known from the prior art. The analysis can thus be carried out, for example, as described by Spackman et al. (Analytical Chemistry, 30, (1958), 1190) by anion exchange chromatography with subsequent ninhydrin derivation, or it can be carried out by reversed phase HPLC, for example as described by Lindroth et al. (Analytical Chemistry (1979) 51: 1167-1174).

The process according to the invention is used for the fermentative preparation of amino acids, in particular L-lysine.

The following microorganism was deposited on 06.02.2001 as a pure culture at the Deutsche Sammlung fur Mikroorganismen und Zellkulturen (DSMZ=German Collection of Microorganisms and Cell Cultures, Braunschweig, Germany) in accordance with the Budapest Treaty:

Corynebacterium glutamicum strain DSM5715ΔotsA as DSM 14041.

The present invention is explained in more detail in the following with the aid of embodiment examples.

The isolation of plasmid DNA from Escherichia coli and all techniques of restriction, Klenow and alkaline phosphatase treatment were carried out by the method of Sambrook et al. (Molecular Cloning. A Laboratory Manual, 1989, Cold Spring Harbor Laboratory Press, Cold Spring Harbor, N.Y., USA). Methods for transformation of Escherichia coli are also described in this handbook.

The composition of the usual nutrient media, such as LB or TY medium, can also be found in the handbook by Sambrook et al.

Having generally described this invention, a further understanding can be obtained by reference to certain specific examples which are provided herein for purposes of illustration only, and are not intended to be limiting unless otherwise specified.

### EXAMPLE 1

### Preparation of a Genomic Cosmid Gene Library from C. glutamicum ATCC 13032

Chromosomal DNA from C. glutamicum ATCC 13032 is isolated as described by Tauch et al. (1995, Plasmid 33:168-179) and partly cleaved with the restriction enzyme Sau3AI (Amersham Pharmacia, Freiburg, Germany, Product Description Sau3AI, Code no. 27-0913-02). The DNA fragments are dephosphorylated with shrimp alkaline phosphatase (Roche Molecular Biochemicals, Mannheim, Germany, Product Description SAP, Code no. 1758250). The DNA of the cosmid vector SuperCos1 (Wahl et al. (1987), Proceedings of the National Academy of Sciences, USA 84:2160-2164), obtained from Stratagene (La Jolla, USA, Product Description SuperCos1 Cosmid Vector Kit, Code no. 251301) is cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA is then cleaved with the restriction enzyme BamHI (Amersham Pharmacia, Freiburg, Germany, Product Description BamHI, Code no. 27-0868-04). The cosmid DNA treated in this manner is mixed with the treated ATCC13032 DNA and the batch is treated with T4 DNA ligase (Amersham Pharmacia, Freiburg, Germany, Product Description T4-DNA-Ligase, Code no.27-0870-04). The ligation mixture is then packed in phages with the aid of Gigapack II XL Packing Extract (Stratagene, La Jolla, USA, Product Description Gigapack II XL Packing Extract, Code no. 200217).

For infection of the E. coli strain NM554 (Raleigh et al. 1988, Nucleic Acids Res. 16:1563-1575) the cells are taken up in 10 mM MgSO₄ and mixed with an aliquot of the phage suspension. The infection and titering of the cosmid library are carried out as described by Sambrook et al. (1989, Molecular Cloning: A laboratory Manual, Cold Spring Harbor), the cells being plated out on LB agar (Lennox, 1955, Virology, 1:190)+100 mg/l ampicillin. After incubation overnight at 37° C., recombinant individual clones are selected.

### EXAMPLE 2

### Isolation and Sequencing of the OtsA Gene

The cosmid DNA of an individual colony is isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments are dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp are isolated with the QiaExII Gel Extraction Kit (Product No. 20021, Qiagen, Hilden, Germany).

The DNA of the sequencing vector pZero-1, obtained from Invitrogen (Groningen, The Netherlands, Product Description Zero Background Cloning Kit, Product No. K2500-01) is cleaved with the restriction enzyme BamHI (Amersham Pharmacia, Freiburg, Germany, Product Description BamHI, Product No. 27-0868-04). The ligation of the cosmid fragments in the sequencing vector pzero-1 is carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). This ligation mixture is then electroporated (Tauch et al. 1994, FEMS Microbiol. Letters, 123:343-7) into the E. coli strain DH5αmcr (Grant, 1990, Proceedings of the National Academy of Sciences, U.S.A., 87:4645-4649). Letters, 123:343-7) and plated out on LB agar (Lennox, 1955, Virology, 1:190) with 50 mg/l zeocin.

The plasmid preparation of the recombinant clones is carried out with a Biorobot 9600 (Product No. 900200, Qiagen, Hilden, Germany). The sequencing is carried out by the dideoxy chain-stopping method of Sanger et al. (1977, Proceedings of the National Academy of Sciences, USA, 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The separation by gel electrophoresis and analysis of the sequencing reaction are carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained are then processed using the Staden program package (1986, Nucleic Acids Research, 14:217-231) version 97-0. The individual sequences of the pZero1 derivatives are assembled to a continuous contig. The computer-assisted coding region analysis is prepared with the XNIP program (Staden, 1986, Nucleic Acids Research 14:217-231).

The resulting nucleotide sequence is shown in SEQ ID No. 1. Analysis of the nucleotide sequence shows an open reading frame of 1485 bp, which is called the otsA gene. The otsA gene codes for a polypeptide of 485 amino acids.

### EXAMPLE 3

### Construction of the Vector pK19mobsacBΔotsA for Deletion of the OtsA Gene

3.1. Cloning of the OtsA Gene in the Vector pUC18

For this, chromosomal DNA is isolated from the strain ATCC13032 by the method of Tauch et al. (1995, Plasmid 33:168-179). On the basis of the sequence of the otsA gene known for C. glutamicum from Example 2, the oligonucleotides described below are chosen for generation of the otsA deletion allele (see also SEQ ID NO: 3 and SEQ ID NO:4):

otsA fwd:
5′-CAC CTA TTC TAA GGA CTT CTT CGA-3′
otsA rev:
5′-ACC AAC CAG GTG GAA TCT GTC A-3′

The primers shown are synthesized by MWG Biotech (Ebersberg, Germany) and the PCR reaction is carried out by the standard PCR method of Innis et al. (PCR Protocols. A Guide to Methods and Applications, 1990, Academic Press) with the Taq-polymerase from Boehringer Mannheim (Germany, Product Description Taq DNA polymerase, Product No. 1 146 165). With the aid of the polymerase chain reaction, the primers allow amplification of a DNA fragment approx. 1.8 kb in size. The product amplified in this way is tested electrophoretically in a 0.8% agarose gel.

The PCR product obtained in then cloned in the vector pUC18 (Amersham Pharmacia Biotech, Cat. No. 27-4949-01) with the Sure Clone Ligation Kit from Amersham Pharmacia Biotech (Freiburg, Germany) in accordance with the manufacturer's instructions. The vector pUC18 was linearized beforehand with the restriction enzyme SmaI.

The E. coli strain DH5αmcr (Grant, 1990, Proceedings of the National Academy of Sciences U.S.A., 87:4645-4649) is then electroporated (Tauch et al. 1994, FEMS Microbiol Letters, 123:343-7) with the ligation batch (Hanahan, In. DNA Cloning. A Practical Approach. Vol. 1, IRL-Press, Cold Spring Habor, N.Y., 1989). Selection of plasmid-carrying cells is made by plating out the transformation batch on LB agar (Sambrook et al., Molecular Cloning: A Laboratory Manual. 2^{nd} Ed., Cold Spring Harbor, N.Y., 1989), which has been supplemented with 25 mg/l ampicillin.

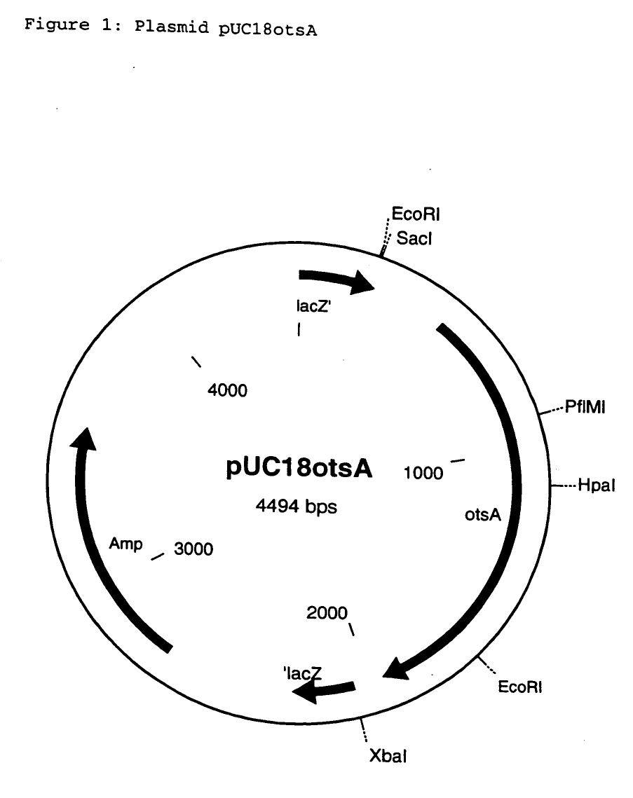

Plasmid DNA is isolated from a transformant with the aid of the QIAprep Spin Miniprep Kit from Qiagen and checked by restriction with the restriction enzyme EcoRI and subsequent agarose gel electrophoresis (0.8%). The plasmid is called pUC18otsA and is shown in FIG. 1.

3.2. Introduction of a Deletion into the Cloned OtsA Gene Fragment

From the plasmid pUC18otsA, a fragment 213 bp in size is excised from the central region of the otsA gene with the restriction enzymes PflMI and HpaI. The 3′ overhangs formed from the PflMI digestion are removed with T4 DNA polymerase (Amersham Pharmacia Biotech, Freiburg, Germany; Code No. E2040Y) in accordance with the manufacturer's instructions. The residual vector is subjected to autoligation with T4 DNA ligase (Amersham Pharmacia Biotech, Freiburg, Germany; Code No. 27-0870-04) in accordance with the manufacturer's instructions and the ligation batch is electroporated (Tauch et al. 1994, FEMS Microbiol Letters, 123:343-7) in the E. coli strain DH5α (Hanahan, In: DNA Cloning. A Practical Approach. Vol. I, IRL-Press, Oxford, Washington D.C., USA). Selection of plasmid-carrying cells is made by plating out the transformation batch on LB agar (Lennox, 1955, Virology, 1:190) with 25 mg/l ampicillin. After incubation overnight at 37° C., recombinant individual clones were selected. Plasmid DNA was isolated from a transformant with the Qiaprep Spin Miniprep Kit (Product No. 27106, Qiagen, Hilden, Germany) in accordance with the manufacturer's instructions and cleaved with the restriction enzyme EcoRI to check the plasmid by subsequent agarose gel electrophoresis. The resulting plasmid is called pUC18AotsA.

3.3. Construction of the Replacement Vector pK19MobsacΔotsA

The otsA deletion allele is isolated by complete cleavage of the vector pUC18ΔotsA, obtained in Example 3.2, with the restriction enzymes SacI/XbaI. After separation in an agarose gel (0.8%), the otsAdel fragment approx. 1.6 kb in size is isolated from the agarose gel with the aid of the Qiagenquick Gel Extraction Kit (Qiagen, Hilden, Germany). The 5′ and 3′ overhangs formed by the restriction digestion are removed with T4 DNA polymerase (Amersham Pharmacia Biotech, Freiburg, Germany; Code No. E2040Y) in accordance with the manufacturer's instructions.

The otsA deletion allele treated in this way is employed for ligation with the mobilizable cloning vector pK19mobsacB (Schäfer et al., Gene 14: 69-73 (1994)). This was cleaved open beforehand with the restriction enzyme SmaI and then dephosphorylated with shrimp alkaline phosphatase (Roche Diagnostics GmbH, Mannheim, Germany, Product No. 1758250). The vector DNA is mixed with the otsA deletion allele and the mixture is treated with T4 DNA ligase (Amersham-Pharmacia, Freiburg, Germany).

The E. coli strain DH5αmcr (Grant, 1990, Proceedings of the National Academy of Sciences U.S.A., 87:4645-4649) is then electroporated with the ligation batch (Hanahan, In. DNA Cloning. A Practical Approach. Vol. 1, IRL-Press, Cold Spring Habor, N.Y., 1989). Selection of plasmid-carrying cells is made by plating out the transformation batch on LB agar (Sambrook et al., Molecular Cloning: A Laboratory Manual. ₂nd Ed., Cold Spring Harbor, N.Y., 1989), which has been supplemented with 25 mg/l kanamycin.

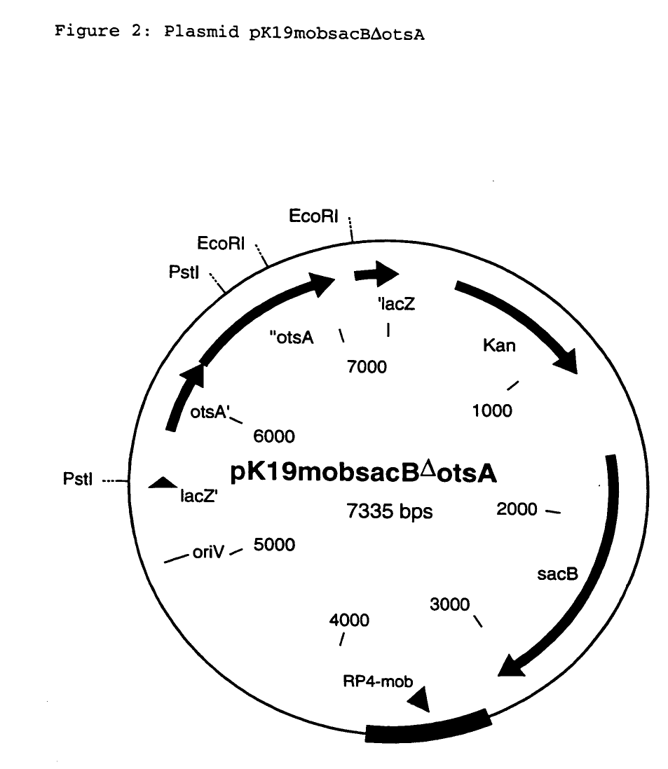

Plasmid DNA is isolated from a transformant with the aid of the QIAprep Spin Miniprep Kit from Qiagen and the cloned otsA deletion allele is verified by means of sequencing by MWG Biotech (Ebersberg, Germany). The plasmid is called pK19mobsacBΔotsA and is shown in FIG. 2.

### EXAMPLE 4

### Deletion Mutagenesis of the OtsA Gene in the C. glutamicum Strain DSM 5715

The vector pK19mobsacBΔotsA mentioned in Example 3.3 is electroporated by the electroporation method of Tauch et al.(1989 FEMS Microbiology Letters 123: 343-347) in Corynebacterium glutamicum DSM5715. The vector cannot replicate independently in DSM5715 and is retained in the cell only if it has integrated into the chromosome. Selection of clones with integrated pK19mobsacBΔotsA takes place by plating out the electroporation batch on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which was supplemented with 15 mg/l kanamycin. Incubation is carried out for 2 days at 33° C.

Clones which have grown on are plated out on LB agar plates with 25 mg/l kanamycin and incubated for 16 hours at 33° C. To achieve excision of the plasmid together with the complete chromosomal copy of the otsA gene, the clones are then grown on LB agar (Sambrook et al., Molecular Cloning: A Laboratory Manual. 2^{nd} Ed., Cold Spring Harbor, N.Y., 1989) with 10% sucrose. The plasmid pK19mobsacB contains a copy of the sacB gene, which converts sucrose into levan sucrase, which is toxic to C. glutamicum. Only those clones in which the pK19mobsacBΔotsA integrated has been excised again therefore grow on LB agar with sucrose. In the excision, together with the plasmid either the complete chromosomal copy of the otsA gene can be excised, or the incomplete copy with the internal deletion. To demonstrate that the incomplete copy of otsA has remained in the chromosome, the plasmid pK9mobsacBΔotsA is marked by the method of “The DIG System Users Guide for Filter Hybridization” from Boehringer Mannheim GmbH (Mannheim, Germany, 1993) using the Dig hybridization kit from Boehringer. Chromosomal DNA of a potential deletion mutant is isolated by the method of Eikmanns et al. (Microbiology 140: 1817-1828 (1994)) and in each case cleaved with the restriction enzymes EcoRI and PstI in separate batches. The fragments formed are separated by agarose gel electrophoresis and hybridized at 68° C. with the Dig hybridization kit from Boehringer. With the aid of the fragments formed, it can be shown that the strain DSM5715 has lost its complete copy of the otsA gene and instead has only the copy with the deletion.

The strain is called C. glutamicum DSM5715ΔotsA and deposited as-a pure culture on Jun. 2, 2001 at the Deutsche Sammlung fur Mikroorganismen und Zellkulturen (DSMZ=German Collection of Microorganisms and Cell Cultures, Braunschweig, Germany) as DSM 14041 in accordance with the Budapest Treaty.

### EXAMPLE 5

### Preparation of Lysine

The C. glutamicum strain DSM5715ΔotsA obtained in Example 4 is cultured in a nutrient medium suitable for the production of lysine and the lysine content in the culture supernatant is determined.

For this, the strain is first incubated on an agar plate with the corresponding antibiotic (brain-heart agar with kanamycin (25 mg/l) for 24 hours at 33° C. Starting from this agar plate culture, a preculture is seeded (10 ml medium in a 100 ml conical flask). The complete medium CgIII is used as the medium for the preculture.

Medium Cg III

| | |
|---|---|
| NaCl | 2.5 g/l |
| Bacto-Peptone | 10 g/l |
| Bacto-Yeast extract | 10 g/l |
| Sucrose (autoclaved separately) | 2% (w/v) |

The pH is brought to pH 7.4

Kanamycin (25 mg/l) is added to this. The preculture is incubated for 16 hours at 33° C. at 240 rpm on a shaking machine. A main culture is seeded from this preculture such that the initial OD (660 nm) of the main culture is 0.1. The medium Cg XII (Keilhauer et al. 1993, Journal of Bacteriology 175:5595-5603) with addition of 0.1 g/l leucine is used for the main culture.

Medium Cg XII

| | |
|---|---|
| MOPS (morpholinopropanesulfonic acid) | 42 g/l |
| Urea | 5 g/l |
| (NH₄)₂SO₄ | 20 g/l |
| KH₂PO₄ | 1 g/l |
| K₂HPO₄ | 1 g/l |
| MgSO₄ * 7 H₂O | 0.25 g/l |
| CaCl₂ * 2 H₂O | 10 mg/l |
| FeSO₄ * 7 H₂O | 10 mg/l |
| MnSO₄ * H₂O | 10 mg/l |
| ZnSO₄ * 7 H₂O | 1 mg/l |
| CuSO₄ | 0.2 mg/l |
| NiCl₂ | 0.02 mg/l |
| Biotin (sterile-filtered) | 0.3 mg/l |
| Leucine (sterile-filtered) | 0.1 g/l |
| Protocatechuic acid (sterile-filtered) | 0.03 mg/l |
| Sucrose (autoclaved separately) | 6% (w/v) |

MOPS and the salt solution are brought to pH 7 and autoclaved. The sterile substrate and vitamin solutions are then added.

Culturing is carried out in a 10 ml volume in a 100 ml conical flask with baffles. Kanamycin (25 mg/l) is added Culturing is carried out at 33° C. and 80% atmospheric humidity.

After 73 hours, the OD is determined at a measurement wavelength of 660 nm with a Biomek 1000 (Beckmann Instruments GmbH, Munich). The amount of lysine formed is determined with an amino acid analyzer from Eppendorf-BioTronik (Hamburg, Germany) by ion exchange chromatography and post-column derivation with ninhydrin detection.

The result of the experiment is shown in Table 1.

TABLE 1

| | OD | Lysine HCl |
|---|---|---|
| Strain | (660 nm) | mM |
| DSM5715 | 8.2 | 39 |
| DSM5715ΔotsA | 8.4 | 52 |

The base pair numbers stated are approximate values obtained in the context of reproducibility.

The abbreviations and designations used have the following meaning:

lacZ′: 5′ terminus of the lacZα gene fragment

′lacZ: 3′ terminus of the lacZα gene fragment

otsA: otsA Gene

Amp: Ampicillin resistance gene

oriV: ColE1-similar origin from pMB1

RP4mob: RP4 mobilization site

Kan: Kanamycin resistance gene

otsA′: 5′ terminal fragment of the pck gene

″otsA: 3′ terminal fragment of the pck gene

sacB: The sacB gene which codes for the protein levan sucrose

EcoRI: Cleavage site of the restriction enzyme EcoRI

HpaI: Cleavage site of the restriction enzyme HpaI

PflMI: Cleavage site of the restriction enzyme PflMI

PstI: Cleavage site of the restriction enzyme PstI

SacI: Cleavage site of the restriction enzyme SacI

XbaI: Cleavage site of the restriction enzyme XbaI

The present application claims priority to German Application No. DE 10103873.9, which was filed on Jan. 30, 2001 and DE 10110760.9, which was filed Mar. 07, 2001, the entire contents of which are incorporated herein by reference.

Obviously, numerous modifications and variations on the present invention are possible in light of the above teachings. It is therefore to be understood that within the scope of the appended claims, the invention may be practiced otherwise than as specifically described herein.

1-45. (Cancelled).

46. A process for preparing L-amino acids, comprising:
culturing a recombinant modified Coryneform bacterium for a time and under conditions suitable for producing L-amino acids, and
collecting the L-amino acids;
wherein the recombinant modified Coryneform bacterium expresses a reduced level of the otsA gene product, which is a trehalose 6-phosphate synthase, compared to an unmodified Coryneform bacterium, or
wherein the recombinant modified Coryneform bacterium expresses a otsA gene product having reduced trehalose 6-phosphate synthase activity compared to the otsA gene product expressed in an unmodified Coryneform bacterium.

47. The process of claim 46, wherein the recombinant modified Coryneform bacterium expresses a reduced level of the otsA gene product, which is a trehalose 6-phosphate synthase, compared to an unmodified Coryneform bacterium.

48. The process of claim 47, wherein the otsA gene product is eliminated in the recombinant modified Coryneform bacterium.

49. The process of claim 47, wherein the expression of the otsA gene product is reduced at least 5% compared to an unmodified Coryneform bacterium.

50. The process of claim 47, wherein the expression of the otsA gene product is reduced at least 10% compared to an unmodified Coryneform bacterium.

51. The process of claim 47, wherein the expression of the otsA gene product is reduced at least 25% compared to an unmodified Coryneform bacterium.

52. The process of claim 47, wherein the expression of the otsA gene product is reduced at least 50% compared to an unmodified Coryneform bacterium.

53. The process of claim 47, wherein the expression of the otsA gene product is reduced at least 75% compared to an unmodified Coryneform bacterium.

54. The process of claim 46, wherein the recombinant modified Coryneform bacterium expresses a otsA gene product having reduced trehalose 6-phosphate synthase activity compared to the otsA gene product expressed in an unmodified Coryneform bacterium.

55. The process of claim 54, wherein the activity of the trehalose 6-phosphate synthase is reduced at least 5% compared to an unmodified Coryneform bacterium.

56. The process of claim 54, wherein the activity of the trehalose 6-phosphate synthase is reduced at least 10% compared to an unmodified Coryneform bacterium.

57. The process of claim 54, wherein the activity of the trehalose 6-phosphate synthase is reduced at least 25% compared to an unmodified Coryneform bacterium.

58. The process of claim 54, wherein the activity of the trehalose 6-phosphate synthase is reduced at least 50% compared to an unmodified Coryneform bacterium.

59. The process of claim 54, wherein the activity of the trehalose 6-phosphate synthase is reduced at least 75% compared to an unmodified Coryneform bacterium.

60. The process of claim 46, wherein said recombinant modified Coryneform bacteria is Corynebacterium glutamicum.

61. The process of claim 46, wherein said recombinant modified Coryneform bacterium is selected from the group consisting of Corynebacterium acteoglutamicum, Corynebacterium acetoacidophilum, Corynebacterium thermoaminogenes, and Brevibacterium flavum.

62. The process of claim 46, wherein said recombinant modified Coryneform bacterium further comprises an increased amount of the products of one or more of the following genes compared to the unmodified Coryneform bacterium: dapA which codes for dihydrodipicolinate synthase, gap which codes for glyceraldehyde 3-phosphate dehydrogenase, eno which codes for enolase, tpi which codes for triose phosphate isomerase, pgk which codes for 3-phosphoglycerate kinase, zwf which codes for glucose 6-phosphate dehydrogenase, pyc which codes for pyruvate carboxylase, mqo which codes for malate-quinone oxidoreductase, lysC which codes for a feed-back resistant aspartate kinase, lysE which codes for lysine export, zwa1 which codes for the Zwa1 protein

63. The process of claim 62, wherein the dapA, gap, eno, tpi, pgk, zwf, pyc, mqo, lysC, lysE, and zwa1 genes are overexpressed in the recombinant modified Coryneform bacterium.

64. The process of claim 46, wherein said recombinant modified Coryneform bacterium further comprises a decreased amount of the products of one or more of the following genes compared to the unmodified Coryneform bacterium: pck gene codes for phosphoenol pyruvate carboxykinase, pgi which codes for glucose 6-phosphate isomerase, poxB which codes for pyruvate oxidase, zwa2 which codes for the Zwa2 protein, fda which codes for fructose 1,6-bisphosphate aldolase, hom which codes for homoserine dehydrogenase, thrB which codes for homoserine kinase and panD which codes for aspartate decarboxylase

65. The process of claim 46, wherein said otsA gene comprises a polynucleotide which hybridizes under stringent conditions to a complement of SEQ ID NO:1 and which encodes a protein having reduced trehalose 6-phosphate synthase activity compared to a protein encoded by SEQ ID NO: 1, and wherein said stringent conditions comprise washing in 5×SSC at a temperature of 68° C.


SEQUENCE LISTING (4 sequences)

SEQ ID NO: 1 - DNA, 3010 bp, Corynebacterium glutamicum
  CDS (884)..(2338)
attgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccctgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcagagattcggtaataaaaggtaaaaatcaacctgcttaggcgtctttcgcttaaatagcgtagaatatcgggtcgatcgcttttaaacactcaggaggatccttgccggccaaaatcacggacactcgtcccaccccagaatcccttcacgctgttgaagaggaaaccgcagccggtgcccgcaggattgttgccacctattctaaggacttcttcgacggcgtcactttgatgtgcatgctcggcgttgaacctcagggcctgcgttacaccaaggtcgcttctgaacacgaggaagctcagccaaagaaggctacaaagcggactcgtaaggcaccagctaagaaggctgctgctaagaaaacgaccaagaagaccactaagaaaactactaaaaagaccaccgcaaagaagaccacaaagaagtcttaagccggatcttatatggatgattccaatagctttgtagttgttgctaaccgtctgccagtggatatgactgtccacccagatggtagctatagcatctcccccagccccggtggccttgtcacggggctttcccccgttctggaacaacatcgtggatgttgggtcggatggcctggaactgtagatgttgcacccgaaccatttcgaacagatacgggtgttttgctgcaccctgttgtcctcactgcaagtgactatgaaggcttctacgagggcttttcaaacgcaacgctgtggcctcttttccacgatctgattgttactccggtgtacaacaccgattggtggcatgcgtttcgggaggtaaacctcaagttcgctgaagccgtgagccaagtggcggcacacggtgccactgtgtgggtgcaggactatcagctgttgctggttcctggcattttgcgccagatgcgccctgatttgaagatcggtttcttcctccacattcccttcccttcccctgatctgttccgtcagctgccgtggcgtgaagagattgttcgaggcatgctgggcgcagatttggtgggattccatttggttcaaaacgcagaaaacttccttgcgttaacccagcaggttgccggcactgccgggtctcatgtgggtcagccggacaccttgcaggtcagtggtgaagcattggtgcgtgagattggcgctcatgttgaaaccgctgacggaaggcgagttagcgtcggggcgttcccgatctcgattgatgttgaaatgtttggggaggcgtcgaaaagcgccgttcttgatcttttaaaaacgctcgacgagccggaaaccgtattcctgggcgttgaccgactggactacaccaagggcattttgcagcgcctgcttgcgtttgaggaactgctggaatccggcgcgttggaggccgacaaagctgtgttgctgcaggtcgcgacgccttcgcgtgagcgcattgatcactatcgtgtgtcgcgttcgcaggtcgaggaagccgtcggccgtatcaatggtcgtttcggtcgcatggggcgtcccgtggtgcattatctacacaggtcattgagcaaaaatgatctccaggtgctgtataccgcagccgatgtcatgctggttacgccttttaaagacggtatgaacttggtggctaaagaattcgtggccaaccaccgcgacggcactggtgctttggtgctgtccgaatttgccggcgcggccactgagctgaccggtgcgtatttatgcaacccatttgatgtggaatccatcaaacggcaaatggtggcagctgtccatgatttgaagcacaatccggaatctgcggcaacgcgaatgaaaacgaacagcgagcaggtctatacccacgacgtcaacgtgtgggctaatagtttcctggattgtttggcacagtcgggagaaaactcatgaaccgcgcacgaatcgcgaccataggcgttcttccgcttgctttactgctggcgtcctgtggttcagacaccgtggaaatgacagattccacctggttggtgaccaatatttacaccgatccagatgagtcgaattcgatcagtaatcttgtcatttcccagcccagcttagattttggcaattcttccctgtctggtttcactggctgtgtgccttttacggggcgtgcggaattcttccaaaatggtgagcaaagctctgttctggatgccgattatgtgaccttgtcttccctggatttcgataaacttcccgatgattgccaaggacaagaactcaaagttcataacgagctggttgatcttctgcctggttcttttgaaatctccaggacttctggttcagaaatcttgctgactagcgatgtcgatgaactcgatcggccagcaatccgcttggtgtcctggatcgcgccgacatcttaaggtgccagggctttaaagtgccaggggttctgtgggatccgtacactggttcccatgactttgactattgaggaaatcgccaagaccaaaaagcttttggttgtgtccgattttgatggaaccatcgcaggatttagcaaggacgcttacaacgttcctatcaaccagaaatccctcaaggcgg

SEQ ID NO: 2 - protein, 485 aa, Corynebacterium glutamicum
MDDSNSFVVVANRLPVDMTVHPDGSYSISPSPGGLVTGLSPVLEQHRGCWVGWPGTVDVAPEPFRTDTGVLLHPVVLTASDYEGFYEGFSNATLWPLFHDLIVTPVYNTDWWHAFREVNLKFAEAVSQVAAHGATVWVQDYQLLLVPGILRQMRPDLKIGFFLHIPFPSPDLFRQLPWREEIVRGMLGADLVGFHLVQNAENFLALTQQVAGTAGSHVGQPDTLQVSGEALVREIGAHVETADGRRVSVGAFPISIDVEMFGEASKSAVLDLLKTLDEPETVFLGVDRLDYTKGILQRLLAFEELLESGALEADKAVLLQVATPSRERIDHYRVSRSQVEEAVGRINGRFGRMGRPVVHYLHRSLSKNDLQVLYTAADVMLVTPFKDGMNLVAKEFVANHRDGTGALVLSEFAGAATELTGAYLCNPFDVESIKRQMVAAVHDLKHNPESAATRMKTNSEQVYTHDVNVWANSFLDCLAQSGENS

SEQ ID NO: 3 - DNA, 24 bp, Corynebacterium glutamicum
cacctattctaaggacttcttcga

SEQ ID NO: 4 - DNA, 22 bp, Corynebacterium glutamicum
accaaccaggtggaatctgtca